(19) United States
(12) Patent Application Publication
(10) Pub. No.: US 2019/0175955 A1
(43) Pub. Date: Jun. 13, 2019

(54) COSMETIC PRODUCT FOR KERATIN-CONTAINING FIBERS CONTAINING AT LEAST ONE SPECIFIC CROSS-LINKED ANIONIC POLYMER AND AT LEAST ONE SPECIFIC CATIONIC POLYMER AND AT LEAST ONE NONIONIC POLYMER

(71) Applicant: Henkel AG & Co. KGaA, Duesseldorf (DE)

(72) Inventors: Diane Metten, Hamburg (DE); Thorsten Knappe, Schenefeld (DE); Marie Meisel, Hemslingen (DE)

(73) Assignee: Henkel AG & Co. KGaA, Duesseldorf (DE)

(21) Appl. No.: 16/214,851

(22) Filed: Dec. 10, 2018

(30) Foreign Application Priority Data
Dec. 11, 2017 (DE) 10 2017 222 396.0

(51) Int. Cl. A61Q 5/06; A61K 8/81; A45D 7/06; A45D 7/04; B65D 83/44

(52) U.S. Cl. CPC A61Q 5/06; A61K 8/817; A61K 8/8147; A61K 2800/87; A45D 7/04; B65D 83/44; A45D 7/06

(57) ABSTRACT

A cosmetic product comprising
A. a dispensing device with spray valve,
B. a container connected to the dispensing device, comprising a cosmetic agent containing
b1) at least one cross-linked anionic polymer (AP) comprising specific monomers,
b2) at least one cationic polymer (KP) comprising at least one structural unit of the formula (I), at least one structural unit of the formula (II) and at least one structural unit of the formula (III),
b3) at least one nonionic polymer (NP),
b4) propellant in a total amount of from about 0 to about 95% by weight, based on the total weight of the cosmetic agent found in the container.

### CROSS-REFERENCE TO RELATED APPLICATION

This application claims priority to German Patent Application No. 10 2017 222 396.0, filed Dec. 11, 2017, which is incorporated herein by reference in its entirety.

### TECHNICAL FIELD

The application relates to the technical field of temporary transformation of keratin-containing fibers, in particular human hair. The subject of the application is cosmetic products containing cosmetic agents containing at least one specific cross-linked anionic polymer and at least one specific (noncross-linked) cationic polymer and at least one (noncross-linked) nonionic polymer, and comprising a dispensing device having a spray valve and the aforementioned cosmetic agents in one container. In addition, the subject of the present application is the use of these cosmetic products for the temporary transformation of keratin-containing fibers and corresponding application methods.

### BACKGROUND

An attractive-looking hairstyle is now generally considered an indispensable part of a well-groomed appearance. Due to current fashion trends, hairstyles are considered to be chic again and again, which in many hair types can only be built up, or can only be maintained for a longer period of time up to several days using consolidating active ingredients. Hair treatment agents which are used for a permanent or temporary shaping of the hair therefore play an important role. While the chemical structure of the keratin-containing fiber is modified by reduction and oxidation during permanent forming, such modifications of the chemical structure do not take place during temporary forming. Corresponding temporary deformation agents usually contain synthetic polymers and/or waxes as a consolidating agent.

The most important property of an agent for the temporary deformation of keratin fibers, also referred to as a styling agent below, is that of treating the treated fibers in the newly modeled form, that is, a form imprinted on the fibers, to give as strong a hold as possible. If the keratin fibers are human hair, it is also known as a strong hairstyle hold or high degree of retention of the styling agent. The hairstyle hold is essentially determined by the type and amount of consolidating active ingredients used, wherein however, an influence of the further constituents of the styling agent and the application form may be provided.

In addition to a high degree of retention, styling agents must meet a whole range of further requirements. These can be roughly subdivided into properties on the hair, properties of the respective formulation, for example, of the sprayed aerosol or non-aerosol, and properties that affect the handling of the styling agent, wherein the properties on the hair is of particular importance. Particularly noteworthy are moisture resistance, low tackiness and a balanced conditioning effect. Furthermore, a styling agent should be universally applicable for all hair types and mild to hair and skin.

In order to meet the different requirements, the prior art has developed a large number of synthetic polymers as consolidating active substances which are used in styling agents. The polymers can be subdivided into cationic, anionic, nonionic and amphoteric consolidating polymers. Ideally, when applied to hair, the polymers give a polymeric film which, on the one hand, gives the hairstyle a strong hold but, on the other hand, is sufficiently flexible not to break under stress. If the polymer film is too brittle, it leads to the formation of so-called film plaques or residues, which detach during the movement of the hair and give the impression that the user of the corresponding styling agent has dandruff. Similar problems arise when waxes find use as a consolidating active ingredient in the styling agent.

Agents for supporting the temporary formation of keratin-containing fibers can be formulated, for example, as a hair spray, hair wax, hair gel or hair foam. In particular, the application by employing a spray device in the form of a spray enjoys high popularity. However, the application by employing a spraying device, depending on the nature of the styling agent, requires additional measures. Thus, for example, the spray application of thickened hair gels in the prior art has up to now only been possible using a specific combination of thickening agent and styling polymer, since only in this way can sufficient nebulization of the hair gel be ensured by employing the spray application. Although the combination of a carbomer and PVP/VA styling polymers used in the prior art can be adequately nebulized, it does not have a satisfactory long-term retention and volume effect and therefore no longer satisfies the consumer's need.

### BRIEF SUMMARY

This disclosure provides a cosmetic product including:
A. a dispensing device with spray valve,
B. a container connected to the dispensing device, comprising a cosmetic agent comprising
b1) at least one cross-linked anionic polymer (AP) comprising at least the following monomers:
bb1) at least one vinylamide monomer A selected from the group of N-vinylpyrrolidone, N-vinylcaprolactam, N-vinylformamide, N-vinylacetamide and N-vinyl-N-methylacetamide,
bb2) at least one acrylic monomer B selected from the group of acrylic acid, methacrylic acid, C₁-C₆ alkyl acrylates, C₁-C₆ alkyl methacrylates, crotonic acid, itaconic acid and maleic acid,
bb3) at least one acrylic monomer C having at least 2 vinyl groups, selected from the group of pentaerythritol triallyl ether, pentaerythritol triacrylate, pentaerythritol tetraacrylate and methylenebisacrylamide,
b2) at least one cationic polymer (KP) comprising at least one structural unit of the formula (I), at least one structural unit of the formula (II) and at least one structural unit of the formula (III)

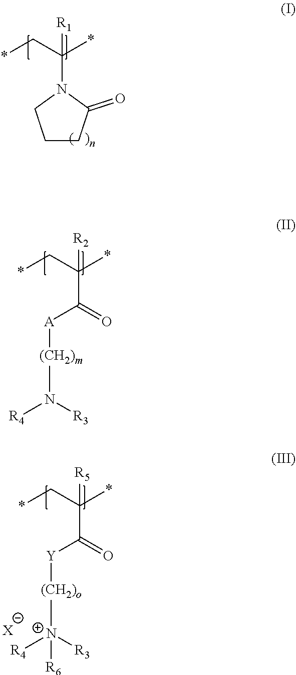

wherein

R¹ , R² and R⁵, each independently of one another, stand for hydrogen or a C₁-C₄ alkyl group,

n, m and o, each independently of one another, stand for integers from about 1 to about 4,

A and Y, each independently of one another, represent an oxygen atom, a sulfur atom or an NH group,

R³ and R⁴, each independently of one another, stand for hydrogen or a C₁-C₄ alkyl group,

R⁶ stands for a linear C₈-C₃₀ alkyl group,

X⁻ stands for a physiologically compatible anion, and

b3) at least one nonionic polymer (NP),

b4) propellant in a total amount of from about 0 to about 95% by weight, based on the total weight of the cosmetic agent found in the container.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was therefore to provide an agent for the temporary deformation of keratin-containing fibers, which are preferably present in the form of a thickened gel and which nevertheless can be applied well to the keratin-containing fibers as a targeted atomized spray. Furthermore, the agent for the temporary deformation keratin-containing fibers should have a high degree of retention, in particular long-term retention, a good hair feel on dry hair and a high volume effect and good styling properties.

It has now surprisingly been found that a combination of a specific cross-linked anionic polymer with a specific (noncross-linked) cationic polymer and a (noncross-linked) nonionic polymer leads to preferably thickened styling agents, which can be well nebulized despite the thickening and can therefore be applied as a fine atomized spray on the keratin fibers. Furthermore, the aforementioned combination of specific polymers leads to a high long-term hold, a good hair feel on dry hair and a good volume effect, that is, a higher volume of the keratin fibers treated with the cosmetic agents as contemplated herein, and good styling properties.

A first subject of the present disclosure is a cosmetic product comprising
A. a dispensing device with spray valve,
B. a container connected to the dispensing device, comprising a cosmetic agent containing
b1) at least one cross-linked anionic polymer (AP) comprising at least the following monomers:
bb1) at least one vinylamide monomer A selected from the group of N-vinylpyrrolidone, N-vinylcaprolactam, N-vinylformamide, N-vinylacetamide and N-vinyl-N-methylacetamide, in particular from N-vinylpyrrolidone,
bb2) at least one acrylic monomer B selected from the group of acrylic acid, methacrylic acid, C₁-C₆ alkyl acrylates, C₁-C₆ alkyl methacrylates, crotonic acid, itaconic acid and maleic acid, in particular from acrylic acid
bb3) at least one acrylic monomer C having at least 2 vinyl groups, selected from the group of pentaerythritol triallyl ether, pentaerythritol triacrylate, pentaerythritol tetraacrylate and methylenebisacrylamide, in particular from pentaerythritol triallyl ether,
b2) at least one cationic polymer (KP) comprising at least one structural unit of the formula (I), at least one structural unit of the formula (II) and at least one structural unit of the formula (III)

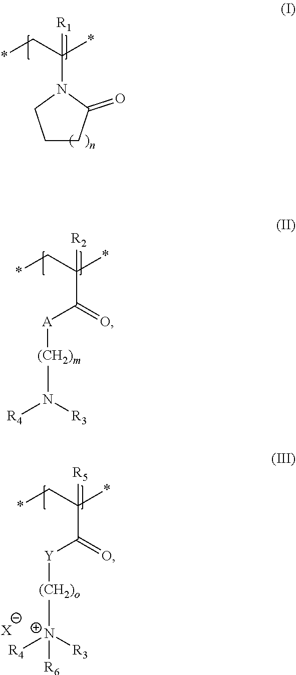

wherein
R¹, R² and R⁵, each independently of one another, stand for hydrogen or a C₁-C₄ alkyl group, n, m and o, each independently of one another, stand for integers from about 1 to about 4,
A and Y, each independently of one another, represent an oxygen atom, a sulfur atom or an NH group, in particular an NH group,
R³ and R⁴, each independently of one another, stand for hydrogen or a C₁-C₄ alkyl group, in particular methyl,
R⁶ stands for a linear C₈-C₃₀ alkyl group, in particular a linear C₁₂ alkyl group,
X⁻ stands for a physiologically compatible anion, in particular chloride, and
b3) at least one nonionic polymer (NP),
b4) propellant in a total amount of from about 0 to about 95% by weight, based on the total weight of the cosmetic agent found in the container.

By combining a specific anionic cross-linked polymer with a specific cationic, preferably noncross-linked, polymer and a preferably noncross-linked, nonionic polymer, thickened, in particular gel-like, cosmetic agents are preferably obtained which, despite the gel-like consistency, can be sprayed or nebulized excellently and therefore can be applied as a fine atomized spray on keratin fibers, in particular human hair. Due to the fine atomization, uniform application of the cosmetic agents as contemplated herein to the keratin fibers is ensured and blockage of the spray valve of the dispensing device is avoided. In addition, the above-mentioned combination of specific cross-linked anionic and cationic and nonionic polymers results in a high long-term hold, a good hair feel on dry hair and a high volume effect and good styling properties.

According to the above formulas and all the following formulas, a chemical bond which is marked with the symbol “*” stands for a free valence of the corresponding structural fragment. Here, free valence is understood to mean the number of atomic bonds which proceeds from the corresponding structural fragment at the position indicated by the symbol “*”. In the context of the present disclosure, an atomic bond in each case preferably proceeds from the positions of the structural fragments marked with the symbol “*” to further structural fragments.

Keratin-containing fibers are in principle understood to mean all animal hair, for example, wool, horsehair, angora hair, furs, feathers and products or textiles made thereof. Preferably, however, the keratin fibers are human hair.

In the context of the present disclosure, the term “anionic polymers” is understood to mean those polymers which carry at least one structural unit having permanent anionic groups in a protic solvent under standard conditions, wherein the anionic groups have to be compensated by counterions while maintaining the electroneutrality. As contemplated herein, anionic groups include, in particular, carboxyl and sulfonic acid groups. In this context, the term “cationic polymers” is understood to mean those polymers which carry at least one structural unit having permanent cationic groups in a protic solvent under standard conditions, wherein the cationic groups must also be compensated by counterions while maintaining the electroneutrality. Permanent cationized groups are understood to mean those groups which form a quaternary ammonium compound. Quaternary ammonium compounds are usually produced by conversion of tertiary amines with alkylating agents, such as methyl chloride, benzyl chloride, dimethyl sulfate, dodecyl bromide, but also ethylene oxide. Depending on the tertiary amine used, the following groups are known in particular: Alkylammonium compounds, alkenylammonium compounds, imidazolinium compounds and pyridinium compounds. Polymers which contain exclusively cationic groups in the form of protonated amines are not included in the “cationic polymers” used in the context of the present disclosure.

In the context of the present disclosure, the term “cross-linked” is understood to mean polymers which have a linkage of the polymer chains by a direct covalent bond of the polymer chains or by bridging molecule fragments, which are each covalently bound to the polymer chains. Cross-linked polymers in the sense of the present disclosure therefore have a network formed by covalent chemical bonds. In the cross-linked anionic polymer (AP), for example, the acrylic monomers C of the component bb3) are corresponding cross-linking monomers.

In the context of the present disclosure, the term “noncross-linked” is understood to mean polymers which do not have a linkage of the polymer chains by a direct covalent bond of the polymer chains or by bridging molecule fragments, which are each covalently bound to the polymer chains. Noncross-linked polymers in the sense of the present disclosure therefore do not have a network formed by covalent chemical bonds.

The agents used as contemplated herein can be sprayed or nebulized.

Furthermore, in the context of the present disclosure, the term “sprayable cosmetic agents” or “nebulizable cosmetic agents” is understood to mean cosmetic agents which can be sprayed using spray devices, in particular aerosol containers or non-aerosol containers with spray valves, and which do not lead to blockage of these valves. As contemplated herein, the nebulization or the spraying of these cosmetic agents by employing spraying devices preferably leads to a fine and targeted atomized spray.

The term “container connected to the dispensing device” is understood in the context of the present disclosure to mean a container from which the cosmetic agent contained therein can enter the spray valve of the dispensing device. Thus, dispensing devices with spray valve and container may be integral, or the dispensing device may be designed with the spray valve, for example, as a screw-on spray head, which may further comprise, for example, a small tube for receiving the cosmetic agent from the container. In this case, the dispensing device with spray valve and the container are formed from at least two pieces. In any case, it is ensured that the cosmetic agents can be transported from the container to the spray valve of the dispensing device.

In addition, the term “physiologically compatible cations” is understood to mean those cations which are non-toxic or non-hazardous or harmless to the organism, in particular the human and the animal organism.

In addition, the term “fatty acid” as used in the context of the present disclosure is understood to mean aliphatic carboxylic acids which have unbranched or branched carbon radicals having from about 4 to about 40 carbon atoms. The fatty acids used in the context of the present disclosure may be both naturally occurring and synthetically produced fatty acids. Furthermore, the fatty acids can be monounsaturated or polyunsaturated.

Finally, the term “fatty alcohol” in the context of the present disclosure is understood to mean aliphatic, monovalent, primary alcohols which have unbranched or branched hydrocarbon radicals having from about 4 to about 40 carbon atoms. The fatty alcohols in the context of the present disclosure may also be monounsaturated or polyunsaturated .

According to a preferred embodiment of this subject, the dispensing device is an aerosol or a non-aerosol container. In the context of the present disclosure, an aerosol container is understood to mean a pressurized gas container filled with propellant, with the aid of which the cosmetic agent as contemplated herein located inside the aerosol container is distributed by the internal gas pressure of the aerosol container via a valve. On the other hand, as contemplated herein, a non-aerosol container is understood to mean a container under normal pressure, with the aid of which the cosmetic agent as contemplated herein located inside the non-aerosol container is distributed as an atomized spray by mechanical action, in particular by a pumping or squeezing system.

The cosmetic product as contemplated herein, which exists in the form of an aerosol spray, can be prepared in a customary manner. As a rule, all constituents of the cosmetic agent as contemplated herein are filled into a suitable pressure-resistant container. This is then closed with a valve. The desired amount of specific propellant is finally filled in by conventional techniques.

Vessels made of metal (aluminum, tinplate, tin), protected or non-chipping plastic or glass, which is coated with plastic on the outside, are regarded as pressure-resistant containers, in which pressure and fracture resistance, corrosion resistance, easy ability to be filled and aesthetic aspects, handiness, ability to be pressurized etc. play a role in their selection. Special internal protective varnishes ensure corrosion resistance with respect to the cosmetic agent as contemplated herein located in the pressure vessel. Particularly preferably, the valves used have an internally varnished valve disk, wherein varnish and valve material are compatible with each other. If aluminum valves are used, their valve plates can be internally coated, for example, with Micoflex varnish. If tinplate valves are used as contemplated herein, their valve disks can be internally be coated with, for example, PET (polyethylene terephthalate). The sizes of the aerosol droplets and the respective size distribution can be set for a given spraying device via the quantity ratio of propellant to the other constituents of the preparations.

The spray rate of the aerosol sprays as contemplated herein is preferably from about 6.5 to about 10.0 g/10 s. Particularly preferred cosmetic products as contemplated herein in the form of an aerosol spray are packaged in an aerosol container with a stem valve having a stem hole from about 0.27 to about 0.35 mm in diameter. Such valves are sold, for example, as valves of the KE or KEN type by Coster.

However, the cosmetic agents used as contemplated herein may also be packaged in a multi-chamber dispensing device. The multi-chamber dispensing device can be used so that one chamber is filled with the compressed propellant and another chamber with the remaining constituents of the cosmetic agent as contemplated herein. Such a multi-chamber dispensing device is, for example, a so-called bag-in-can packaging.

The cosmetic agents used as contemplated herein in the form of non-aerosols or atomized spray formulations can be applied in any propellant-free spray system which has a dispensing container and a spray valve, that is, for example, in a flexible pressure bottle with dip tube and spray valve (squeeze bottle), in a balloon atomizer, which operates according to the Venturi principle or in a pump spray bottle, the pump lever of which is actuated with the index finger or with the whole hand in the manner of a trigger. In a preferred embodiment of the cosmetic product in the form of a non-aerosol for cosmetic use, the dispensing container has a manually actuated spray pump.

Particularly preferred cosmetic products contained a container in the form of a non-aerosol container.

As a first essential constituent a), the cosmetic agent as contemplated herein contains at least one specific cross-linked anionic polymer based on the structural units which are derived from the monomers bb1), bb2) and bb3) by polymerization. Thus, the cross-linked anionic polymers (AP) have at least one vinylamide monomer A, at least one acrylic monomer B and at least one acrylic monomer C.

The vinylamide monomer A is selected from the group of N-vinylpyrrolidine, N-vinylcaprolactam, N-vinylformamide, N-vinylacetamide and N-vinyl-N-methylacetamide, in particular N-vinylpyrrolidone.

Component bb1), the at least one vinylamide monomer A), in particular N-vinylpyrrolidone, is preferably present in the anionic polymer (AP) in a total amount of from about 25 to about 75% by weight, preferably from about 30 to about 70% by weight, particularly preferably from about 35 to about 65% by weight, in particular from about 40 to about 55% by weight, in each case based on the total weight of all monomers of the anionic polymer (AP), or is contained polymerized in this amount in it.

Component bb2), the at least one acrylic monomer B, in particular acrylic acid, is preferably present in the anionic polymer (AP) in a total amount of from about 25 to about 75% by weight, preferably from about 30 to about 70% by weight, particularly preferably from about 35 to about 65% by weight, in particular from about 40 to about 55% by weight, each based on the total weight of all monomers of the anionic polymer (AP).

The component bb3), the at least one acrylic monomer C having at least two vinyl groups, in particular pentaerythritol triallyl ether, is preferably present in the anionic polymer (AP) in a total amount of from about 0.5 to about 3.0% by weight, preferably from about 0.6 to about 2.5% by weight, particularly preferably from about 0.7 to about 2.0% by weight, in particular from about 0.8 to about 1.5% by weight, each based on the total weight of all monomers of the anionic polymer (AP).

Preference is given to a combination of all three preferred monomers A, B and C in the amounts indicated in each case.

The at least one cross-linked anionic polymer (AP) cannot be present neutralized, in partially neutralized or completely neutralized form.

The at least one cross-linked anionic polymer (AP) preferably has a degree of neutralization of from about 40 to about 100%, preferably from about 45 to about 100%, particularly preferably from about 60 to about 100%, in particular from about 80 to about 100%.

An example of a particularly preferred anionic polymer (AP) is a copolymer of 2-propenoic acid with 1-ethylenyl-2-pyrrolidinone and 3-(2-propenyloxy)-2,2-bis((2-propenyloxy)methyl)-1-propanol.

The at least one cross-linked anionic polymer (AP) in the cosmetic agent is preferably present in a total amount of from about 0.1 to about 1.0% by weight, preferably from about 0.1 to about 0.8% by weight, particularly preferably from about 0.1 to about 0.7% by weight, in particular from about 0.2 to about 0.4% by weight, each based on the total weight of the cosmetic agent.

The cosmetic agent used as contemplated herein contains at least one cationic polymer (KP) as a component b2), comprising at least one structural unit of the formula (I), at least one structural unit of the formula (II) and at least one structural unit of the formula (III) as are specified above.

In the structural unit of the formula (I), the radical R¹ preferably stands for hydrogen and n for the integer 3.

The structural unit of the formula (I) is preferably present in the cationic polymer (KP) in a total amount of from about 5 to about 85% by weight, preferably from about 15 to about 82% by weight, particularly preferably from about 25 to about 80% by weight, in particular from about 50 to about 80% by weight, in each case based on the total weight of all monomers of the cationic polymer (KP).

In the structural unit of the formula (II), the radical R² preferably stands for a methyl group, A for an NH group, m for the integer 3, and R³ and R⁴ each for a methyl group.

The structural unit of the formula (II) is preferably present in the cationic polymer (KP) in a total amount of from about 0.05 to about 20% by weight, preferably from about 0.06 to about 15% by weight, particularly preferably from about 0.07 to about 10% by weight, in particular from about 0.08 to about 6% by weight, in each case based on the total weight of all monomers of the cationic polymer (KP).

In the structural unit of the formula (III), A preferably stands for an NH group, o for the integer 3, R³, R⁴ and R⁵ each for a methyl group, R⁶ for a linear C₁₂ alkyl group and X⁻ for chloride.

The structural unit of the formula (III) is preferably present in the cationic polymer (KP) in a total amount of from about 0.1 to about 50% by weight, preferably from about 0.2 to about 40% by weight, particularly preferably from about 0.3 to about 30% by weight, in particular from about 0.4 to about 15% by weight, in each case based on the total weight of all monomers of the cationic polymer (KP).

Preferably, the above-listed structural units of the formulas (I), (II) and (III) are present in the cationic polymer (KP) in the preferred mentioned amounts. Further structural units may be present in addition to the structural units of the formulas (I), (II) and (III). According to one embodiment, however, no further structural units of the formulas (I), (II) and (III) are present in the cationic polymer (KP). In this case, the total amount of the structural units of the formulas (I) to (III) is about 100% by weight.

According to a preferred embodiment of the present disclosure, the cationic polymer (KP) additionally contains at least one structural unit of the formula (IV)

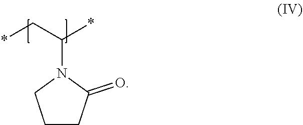

The structural unit of the formula (I) is preferably present in the cationic polymer (KP) in a total amount of from about 5.0 to about 85% by weight, preferably from about 8.0 to about 70% by weight, particularly preferably from about 10 to about 60% by weight, in particular from about 15 to about 45% by weight, in each case based on the total weight of all monomers of the cationic polymer (KP).

If only the structural units of the formula (I) to (IV) are present in the cationic polymer (KP), their total amount yields about 100% by weight. However, further structural units may be present so that the total amount of all structural units is then about 100% by weight.

In the cosmetic agent used as contemplated herein, the at least one cationic polymer (KP) of the component b2) is preferably present in a total amount of from about 0.5 to about 5.0% by weight, preferably from about 0.8 to about 4.0% by weight, in particular preferably from about 1.0 to about 3.0% by weight, in particular from about 1.5 to about 2.5% by weight, each based on the total weight of the cosmetic agent.

An example of a cationic polymer (KP) suitable as contemplated herein is Polyquarternium-69 (INCI). It is a tetrapolymer made from vinylcaprolactam, vinylpyrrolidone, N-(3-dimethylaminopropyl) methacrylamide and 3-methacryloylamino) propyllauryldimethylammonium chloride as basic building blocks. The molecular weight (Mn or Mw, preferably Mn) is preferably from about 100,000 to about 500,000, more preferably from about 200,000 to about 400,000, in particular about 350,000. The viscosity is preferably from about 80,000 to about 100,000, more preferably from about 90,000 to about 100,000, in particular about 97,000, determined with a Brookfield RVT viscometer with TC spindle at ten revolutions per minute. The viscosity is specified in cps.

Any nonionic polymers are suitable as nonionic polymers (NP) of component b3).

The at least one nonionic polymer (NP) of component b3) is preferably selected from the group of homopolymers of polyvinylpyrrolidone, homopolymers of polyvinyl acetate, homopolymers and copolymers of vinyl alcohol, copolymers of N-vinylpyrrolidone and vinyl acetate and mixtures thereof. Particular preference is given to using copolymers which have repeating units of N-vinylpyrrolidone and vinyl acetate. N-vinylpyrrolidone-vinyl acetate copolymers are used in particular.

In this context, it may be provided as contemplated herein in particular that the at least one film-forming and/or consolidating polymer (NP) is selected from copolymers of polyvinylpyrrolidone and vinyl acetate. In particular, polyvinylpyrrolidone / vinyl acetate copolymers having a molar ratio of polyvinylpyrrolidone to vinyl acetate of from about 70 to about 30, from about 60 to about 40, from about 50 to about 50 or from about 30 to about 70 are usable in the context of the present disclosure.

The cosmetic agent contains the at least one nonionic polymer (NP) of component b3), preferably in a total amount of from about 0.5 to about 5.0% by weight, preferably from about 0.6 to about 4.0% by weight, more preferably from about 0.7 to about 3.0% by weight, in particular from about 1.0 to about 2.0% by weight, each based on the total weight of the cosmetic agent.

Particularly preferably, the amount specifications for an N-vinylpyrrolidone-vinyl acetate copolymer are applicable.

The preferred amounts of components b1), b2) and b3) are preferably combined, in particular with the polymers specified as being preferred in each case.

The cosmetic agent used as contemplated herein may still contain further ingredients.

Preferably, the cosmetic agent contains at least one alkalizing agent. “Alkalizing agent” is understood to mean chemical compounds which shift the pH value of the cosmetic agent in the direction of higher pH values. For example, it is possible to use organic or inorganic bases or mixtures thereof

According to a preferred embodiment, the cosmetic agent additionally contains at least one alkalizing agent selected from the group of organic bases such as alkanolamines and inorganic bases such as sodium hydroxide, potassium hydroxide, lithium hydroxide or mixtures thereof

Preferably, the cosmetic agent contains at least one alkalizing agent, in particular alkanolamine, in a total amount of from about 0.05 to about 0.5% by weight, preferably from about 0.06 to about 0.4% by weight, particularly preferably from about 0.07 to about 0.3% by weight, in particular from about 0.1 to about 0.2% by weight, each based on the total weight of the cosmetic agent.

The alkanolamine may be, for example, 2-amino-2-methyl-1-propanol.

The cosmetic agent used as contemplated herein may be an aerosol or non-aerosol. Accordingly, the container may be an aerosol container or preferably a non-aerosol container.

If the cosmetic agent as contemplated herein is present as an aerosol spray, it preferably contains at least one propellant in a total amount of from about 10 to about 80% by weight, preferably from about 20 to about 70% by weight, in particular from about 30 to about 60% by weight, based on the total weight of the cosmetic agent. Preferably, therefore, the cosmetics do not contain water.

Propellants suitable as contemplated herein are selected, for example, from N₂O, dimethyl ether, CO2, air, alkanes having from about 3 to about 5 carbon atoms, such as propane, n-butane, isobutane, n-pentane and isopentane, and mixtures thereof. Preference is given to dimethyl ether, propane, n-butane, isobutane and mixtures thereof. According to a preferred embodiment, the alkanes mentioned, mixtures of said alkanes or mixtures of said alkanes with dimethyl ether are used as the sole propellant. However, the present disclosure expressly also includes the concomitant use of propellants of the type of chlorofluorohydrocarbons, but in particular of fluorocarbons.

The propellant (in particular dimethyl ether) is preferably present in the agents as contemplated herein of the embodiment as an aerosol spray in a total amount of from about 30 to about 60% by weight, based on the total weight of the cosmetic agent.

Very particular preference is given to using dimethyl ether or mixtures of propane and butane as the sole propellant in a weight ratio of propane to butane of from about 20 to about 80 up to from about 15 to about 85. The mixtures are again preferably used in the agents as contemplated herein in a total amount of from about 30 to about 55% by weight, based on the total weight of the cosmetic agent. Butane as contemplated herein is understood to mean n-butane, isobutane and mixtures of n-butane and isobutane. Most preferably, dimethyl ether is used as the sole propellant.

If it is a non-aerosol, the cosmetic agent used as contemplated herein preferably contains no propellant, but rather larger amounts of water. With particular regard to the cosmetic agents used as contemplated herein ability to be sprayed, it is advantageous when the cosmetic agent contains water in a total amount of from about 75 to about 98.9% by weight, preferably from about 80 to about 98% by weight, particularly preferably from about 85 to about 90% by weight, in particular from about 86 to about 88% by weight, based on the total weight of the cosmetic agent.

Typically, in addition to the other ingredients, the amount of water is adjusted so that the total amount of the cosmetic agent is about 100% by weight.

In the context of this embodiment, however, it can also be provided that the cosmetic agent as contemplated herein further contains at least one liquid solvent other than water, liquid at about 25° C. and about 1013 mbar in a total amount of about 1% by weight to about 50% by weight, preferably from about 5% by weight to about 40% by weight, in particular from about 10% by weight to about 30% by weight, based on the total weight of the cosmetic agent.

It may be preferred as contemplated herein when at least one (C₂ to C₆) alkyl alcohol having at least one hydroxyl group is used as an additional solvent of the agent as contemplated herein. In the context of a particularly preferred embodiment of the agent as contemplated herein, the agent contains as an additional solvent at least one alcohol which has from about 2 to about 6 carbon atoms and from about 1 to about 3 hydroxyl groups.

The solvent other than water is preferably selected from at least one compound of the group which is formed from ethanol, ethylene glycol, isopropanol, 1,2-propylene glycol, 1,3-propylene glycol, glycerol, n-butanol, 1,3-butylene glycol. A most preferred solvent is ethanol.

Further, particularly preferred solvents are polyethylene glycol and/or polypropylene glycol.

In particular, the addition of polyethylene glycol and/or polypropylene glycol increases the flexibility of the polymer film formed when using the cosmetic agent as contemplated herein. Thus, if a flexible hold is desired, the cosmetic agents as contemplated herein preferably contain a total amount of from about 0.01 to about 30% by weight of polyethylene glycol and/or polypropylene glycol, based on the total weight of the cosmetic agent.

According to a particularly preferred embodiment of the present disclosure, the cosmetic agent is present as a gel. In the context of the present disclosure, the term “gel” is understood to mean a dimensionally stable and easily deformable system made of two components, wherein the one component forms a spatial, three-dimensional network in the form of the thickening agent or gelling agent, in the spaces or pores of said network is incorporated the second component in the form of liquid, in particular water.

The cosmetic agents as contemplated herein according to this embodiment preferably have a viscosity of from about 2,500 to about 7,000 mPas, more preferably from about 3,000 to about 6,000 mPas, in particular from about 3,500 to about 5,000 mPas, each measured with Brookfield RVDV II+ with Heilpath, spindle 2, about 5 rpm, about 20° C. Cosmetic agents as contemplated herein, which have the aforementioned viscosity, despite their thickened consistency, have an excellent nebulization and do not lead to an adhesion of the spray valves of the dispensing device.

In addition to the polymers b1), b2) and b3) described above, the cosmetic agents used as contemplated herein may contain further ingredients. The group of these further ingredients includes in particular cosmetically active auxiliaries and additives, in particular further polymers and additional care substances.

The long-term hold of the cosmetic agent as contemplated herein can optionally be further increased when the cosmetic agent contains, in addition to component b3), at least one further film-forming and/or consolidating polymer. Film-forming or consolidating polymers contribute to the hold of the imprinted form of the fiber collective, for example, the total hairstyle, by film formation. The film formation can be quite point-oriented and connect only a few fibers with each other. Film-forming or consolidating polymers are understood to mean those polymers which leave a continuous film on the skin, hair or nails with drying. Such film formers can be used in a wide variety of cosmetic products, such as, face masks, make-up, hair fixatives, hair sprays, hair gels, hair waxes, hair treatments, shampoos or nail varnishes. Particularly preferred are those polymers which have sufficient solubility in water or water/alcohol mixtures in order to be present in completely dissolved form in the cosmetic agents as contemplated herein. The film-forming polymers may be of synthetic or natural origin. Film-forming polymers are further understood to mean those polymers which are capable of depositing a transparent polymer film on the hair when applied in from about 0.01 to about 20% by weight aqueous, alcoholic or aqueous alcoholic solution.

In addition to, for example, the copolymers of polyvinylpyrrolidone and vinyl acetate of component b3), the cosmetic agents as contemplated herein may contain further film-forming and/or consolidating polymers selected from the group of nonionic film-forming and/or consolidating polymers, amphoteric film-forming and/or consolidating polymers and mixtures thereof

In the context of the present disclosure, nonionic polymers are understood to mean polymers which have no permanent anionic or permanent cationic groups and no anionizable or cationizable groups, such as, for example, carboxylic acid groups or amine groups. As contemplated herein, such film-forming and/or consolidating nonionic polymers having at least one structural element of the formula (VIII)

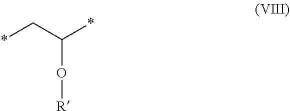

are preferably suitable, which, according to formula (VIII), carry a hydrogen atom, an acetyl group or a propanoyl group, in particular an acetyl group as R′.

The consolidating nonionic polymers are again preferably selected from at least one polymer of the group which is formed from
copolymers of N-vinylpyrrolidone and N-vinylimidazole and methacrylamide,
copolymers of N-vinylpyrrolidone and N-vinylimidazole and acrylamide,
copolymers of N-vinylpyrrolidone with N,N-di(C₁ to C₄)-alkylamino-(C₂ to C₄)-alkylacrylamide,
copolymers of N-vinylpyrrolidone with N,N-di(C₁ to C₄)-alkylamino-(C₂ to C₄)-alkylacrylamide,
nonionic copolymers of isobutene.

The cosmetic agents as contemplated herein may also contain, as a further film-forming and/or consolidating polymer, at least one film-forming and/or consolidating amphoteric polymer. The term amphoteric polymers are understood to mean, on the one hand, polymers which contain both free amino groups and free —COOH or SO₃H groups in the molecule and are capable of forming internal salts. On the other hand, zwitterionic polymers according to the present disclosure include such polymers which contain in the molecule, quaternary ammonium groups and —COO⁻ or —SO₃⁻ groups or quaternary ammonium groups and —COOH or SO₃H groups.

An example of a usable film-forming and/or consolidating amphoteric polymer as contemplated herein is the acrylic resin available under the name Amphomer®, which is a copolymer of tert-butylaminoethyl methacrylate, N-(1,1,3,3-tetramethylbutyl) acrylamide and two or more monomers from the group of acrylic acid, methacrylic acid and their (C₁ to C₄) alkyl esters. These polymers have, in addition to the cationogenic group or positively charged group, at least one negatively charged group in the molecule and are also referred to as zwitterionic polymers.

The additional film-forming and/or consolidating polymers may preferably be present in the cosmetic agents as contemplated herein in an amount of from about 0.1% by weight to about 12.0% by weight, preferably from about 0.2% by weight to about 10.0% by weight, in particular from about 0.5% by weight to about 8.0% by weight, in each case based on the total weight of the cosmetic agent.

However, particularly preferred cosmetic agents contain no further polymers, apart from the abovementioned polymers b1) to b3). Such agents are therefore exemplified in that they, apart from the polymers b1) to b3), contain additional polymers in a total amount of from about 0 to about 0.5% by weight, in particular of about 0% by weight, each based on the total weight of cosmetic agent.

For example, a silicone oil and/or a silicone gum can be used as a care substance. Silicone oils or silicone gums suitable as contemplated herein are, in particular, dialkyl and alkylaryl siloxanes, such as dimethylpolysiloxane and methylphenylpolysiloxane, and also their alkoxylated, quaternized or else anionic derivatives. Preference is given to cyclic and linear polydialkylsiloxanes, their alkoxylated and/or aminated derivatives, dihydroxypolydimethylsiloxanes and polyphenylalkylsiloxanes, in particular PEG-12 dimethicones and PEG-14 dimethicones.

The cosmetic agent as contemplated herein may contain as a care substance of another class of compounds, for example, at least one protein hydrolyzate and/or one of its derivatives. Protein hydrolyzates are product mixtures which are obtained by acid, alkaline or enzymatically catalyzed degradation of proteins. As contemplated herein, the term “protein hydrolyzates” are also understood to mean total hydrolyzates of individual amino acids and their derivatives and mixtures of different amino acids. The molecular weight of the protein hydrolysates which can be used as contemplated herein is between about 75, the molecular weight for glycine, and about 200,000 daltons; the molecular weight is preferably from about 75 to about 50,000 daltons, in particular from about 75 to about 20,000 daltons.

The cosmetic agent used as contemplated herein may further contain as a care substance at least one vitamin, one provitamin, one vitamin precursor and/or derivatives thereof. As contemplated herein, such vitamins, provitamins and vitamin precursors which are usually assigned to the groups A, B, C, E, F and H are preferred.

As with the addition of glycerol and/or propylene glycol, the addition of panthenol increases the flexibility of the polymer film formed when applying the cosmetic agent used as contemplated herein.

The cosmetic agents used as contemplated herein may further contain as a care substance at least one plant extract, but also mono- or oligosaccharides and/or lipids.

Furthermore, oil bodies are suitable as a care substance. The natural and synthetic cosmetic oil bodies include, for example, vegetable oils, liquid paraffin oils, isoparaffin oils and synthetic hydrocarbons, and di-n-alkyl ethers having a total of between from about 12 and about 36 carbon atoms, in particular from about 12 to about 24 carbon atoms.

Ester oils, that is, esters of C₆-C₃₀ fatty acids with C₂-C₃₀ fatty alcohols, preferably monoesters of fatty acids with alcohols having from about 2 to about 24 carbon atoms such as isopropyl myristate (Rilanit® IPM), isononanoic acid C6-18 alkyl ester (Cetiol® SN), 2-ethylhexyl palmitate (Cegesoft® 24), stearic acid-2-ethylhexyl ester (Cetiol® 868), cetyl oleate, glycerol tricaprylate, cocofatty alcohol caprate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), Cetearyl Isononanoate (Cetiol® SN), oleic acid decyl ester (Cetiol® V) are further preferred caring oil bodies.

Dicarboxylic acid esters, symmetrical, unsymmetrical or cyclic esters of carbonic acid with fatty alcohols, triflic acid esters of saturated and/or unsaturated linear and/or branched fatty acids with glycerol or fatty acid partial glycerides, which are to be understood to mean monoglycerides, diglycerides and their technical mixtures, are suitable as care substances.

In the following tables are particularly preferred embodiments AF1 to AFx of the cosmetic agents contained in the cosmetic products as contemplated herein (all data in % by weight based on the total weight of the cosmetic agent):

| | AF 1 | AF 2 | AF 3 | AF 4 |
|---|---|---|---|---|
| Cross-linked anionic | 0.1 to 1.0 | 0.1 to 0.8 | 0.1 to 0.7 | 0.2 to 0.4 |
| polymer (AP)¹⁾ | | | | |
| Cationic polymer (KP)²⁾ | 0.5 to 5.0 | 0.8 to 4.0 | 1.0 to 3.0 | 1.5 to 2.5 |
| Nonionic polymer (NP) | 0.5 to 5.0 | 0.6 to 4.0 | 0.7 to 3.0 | 1.0 to 2.0 |
| Propellant | 0 to 95 | 0 to 95 | 0 to 95 | 0 |
| | AF 5 | AF 6 | AF 7 | AF 8 |
| Cross-linked anionic | 0.1 to 1.0 | 0.1 to 0.8 | 0.1 to 0.7 | 0.2 to 0.4 |
| polymer (AP)³⁾ | | | | |
| Cationic polymer (KP)²⁾ | 0.5 to 5.0 | 0.8 to 4.0 | 1.0 to 3.0 | 1.5 to 2.5 |
| Nonionic polymer (NP) | 0.5 to 5.0 | 0.6 to 4.0 | 0.7 to 3.0 | 1.0 to 2.0 |
| Propellant | 0 to 95 | 0 to 95 | 0 to 95 | 0 |
| | AF 9 | AF 10 | AF 11 | AF 12 |
| Cross-linked anionic | 0.1 to 1.0 | 0.1 to 0.8 | 0.1 to 0.7 | 0.2 to 0.4 |
| polymer (AP)⁴⁾ | | | | |
| Cationic polymer (KP)²⁾ | 0.5 to 5.0 | 0.8 to 4.0 | 1.0 to 3.0 | 1.5 to 2.5 |
| Nonionic polymer (NP) | 0.5 to 5.0 | 0.6 to 4.0 | 0.7 to 3.0 | 1.0 to 2.0 |
| Propellant | 0 to 95 | 0 to 95 | 0 to 95 | 0 |
| | AF 13 | AF 14 | AF 15 | AF 16 |
| Cross-linked anionic | 0.1 to 1.0 | 0.1 to 0.8 | 0.1 to 0.7 | 0.2 to 0.4 |
| polymer (AP)¹⁾ | | | | |
| Cationic polymer (KP)⁵⁾ | 0.5 to 5.0 | 0.8 to 4.0 | 1.0 to 3.0 | 1.5 to 2.5 |
| Nonionic polymer (NP) | 0.5 to 5.0 | 0.6 to 4.0 | 0.7 to 3.0 | 1.0 to 2.0 |
| Propellant | 0 to 95 | 0 to 95 | 0 to 95 | 0 |
| | AF 17 | AF 18 | AF 19 | AF 20 |
| Cross-linked anionic | 0.1 to 1.0 | 0.1 to 0.8 | 0.1 to 0.7 | 0.2 to 0.4 |
| polymer (AP)¹⁾ | | | | |
| Cationic polymer (KP)⁶⁾ | 0.5 to 5.0 | 0.8 to 4.0 | 1.0 to 3.0 | 1.5 to 2.5 |
| Nonionic polymer (NP) | 0.5 to 5.0 | 0.6 to 4.0 | 0.7 to 3.0 | 1.0 to 2.0 |
| Propellant | 0 to 95 | 0 to 95 | 0 to 95 | 0 |
| | AF 21 | AF 22 | AF 23 | AF 24 |
| Cross-linked anionic | 0.1 to 1.0 | 0.1 to 0.8 | 0.1 to 0.7 | 0.2 to 0.4 |
| polymer (AP)⁴⁾ | | | | |
| Cationic polymer (KP)⁶⁾ | 0.5 to 5.0 | 0.8 to 4.0 | 1.0 to 3.0 | 1.5 to 2.5 |
| Nonionic polymer (NP) | 0.5 to 5.0 | 0.6 to 4.0 | 0.7 to 3.0 | 1.0 to 2.0 |
| Propellant | 0 to 95 | 0 to 95 | 0 to 95 | 0 |
| | AF 25 | AF 26 | AF 27 | AF 28 |
| Cross-linked anionic | 0.1 to 1.0 | 0.1 to 0.8 | 0.1 to 0.7 | 0.2 to 0.4 |
| polymer (AP)¹⁾ | | | | |
| Cationic polymer (KP)²⁾ | 0.5 to 5.0 | 0.8 to 4.0 | 1.0 to 3.0 | 1.5 to 2.5 |
| Nonionic polymer (NP)⁷⁾ | 0.5 to 5.0 | 0.6 to 4.0 | 0.7 to 3.0 | 1.0 to 2.0 |
| Propellant | 0 to 95 | 0 to 95 | 0 to 95 | 0 |
| | AF 29 | AF 30 | AF 31 | AF 32 |
| Cross-linked anionic | 0.1 to 1.0 | 0.1 to 0.8 | 0.1 to 0.7 | 0.2 to 0.4 |
| polymer (AP)³⁾ | | | | |
| Cationic polymer (KP)²⁾ | 0.5 to 5.0 | 0.8 to 4.0 | 1.0 to 3.0 | 1.5 to 2.5 |
| Nonionic polymer (NP)⁷⁾ | 0.5 to 5.0 | 0.6 to 4.0 | 0.7 to 3.0 | 1.0 to 2.0 |
| Propellant | 0 to 95 | 0 to 95 | 0 to 95 | 0 |
| | AF 33 | AF 34 | AF 35 | AF 36 |
| Cross-linked anionic | 0.1 to 1.0 | 0.1 to 0.8 | 0.1 to 0.7 | 0.2 to 0.4 |
| polymer (AP)⁴⁾ | | | | |
| Cationic polymer (KP)²⁾ | 0.5 to 5.0 | 0.8 to 4.0 | 1.0 to 3.0 | 1.5 to 2.5 |
| Nonionic polymer (NP)⁷⁾ | 0.5 to 5.0 | 0.6 to 4.0 | 0.7 to 3.0 | 1.0 to 2.0 |
| Propellant | 0 to 95 | 0 to 95 | 0 to 95 | 0 |
| | AF 37 | AF 38 | AF 39 | AF 40 |
| Cross-linked anionic | 0.1 to 1.0 | 0.1 to 0.8 | 0.1 to 0.7 | 0.2 to 0.4 |
| polymer (AP)¹⁾ | | | | |
| Cationic polymer (KP)⁵⁾ | 0.5 to 5.0 | 0.8 to 4.0 | 1.0 to 3.0 | 1.5 to 2.5 |
| Nonionic polymer (NP)⁷⁾ | 0.5 to 5.0 | 0.6 to 4.0 | 0.7 to 3.0 | 1.0 to 2.0 |
| Propellant | 0 to 95 | 0 to 95 | 0 to 95 | 0 |
| | AF 41 | AF 42 | AF 43 | AF 44 |
| Cross-linked anionic | 0.1 to 1.0 | 0.1 to 0.8 | 0.1 to 0.7 | 0.2 to 0.4 |
| polymer (AP)¹⁾ | | | | |
| Cationic polymer (KP)⁶⁾ | 0.5 to 5.0 | 0.8 to 4.0 | 1.0 to 3.0 | 1.5 to 2.5 |
| Nonionic polymer (NP)⁷⁾ | 0.5 to 5.0 | 0.6 to 4.0 | 0.7 to 3.0 | 1.0 to 2.0 |
| Propellant | 0 to 95 | 0 to 95 | 0 to 95 | 0 |
| | AF 45 | AF 46 | AF 47 | AF 48 |
| Cross-linked anionic | 0.1 to 1.0 | 0.1 to 0.8 | 0.1 to 0.7 | 0.2 to 0.4 |
| polymer (AP)⁴⁾ | | | | |
| Cationic polymer (KP)⁶⁾ | 0.5 to 5.0 | 0.8 to 4.0 | 1.0 to 3.0 | 1.5 to 2.5 |
| Nonionic polymer (NP)⁷⁾ | 0.5 to 5.0 | 0.6 to 4.0 | 0.7 to 3.0 | 1.0 to 2.0 |
| Propellant | 0 to 95 | 0 to 95 | 0 to 95 | 0 |

¹⁾contains the previously defined monomers bb1) to bb3),
²⁾contains the previously defined structural units of the formulas (I) to (III),
³⁾containing N-vinylpyrrolidone as vinylamide monomer A, acrylic acid as acrylic monomer B and pentaerythritol triallyl ether as acrylic monomer C,
⁴⁾containing from about 40 to about 55% by weight N-vinylpyrrolidone, from about 40 to about 55% by weight acrylic acid and from about 0.8 to about 1.5% by weight pentaerythritol triallyl ether, each based on the total weight of all monomers of the cross-linked anionic polymer (AP),
⁵⁾containing structural units of formulas (I) to (III) with R₁ = H, R², R³, R⁴ and R⁵, each independently of one another = CH₃, R⁶ = linear C₁₂ alkyl group, X⁻ = chloride, A = NH, m, n and o each independently of one another = 3,
⁵⁾containing structural units of formulas (I) to (IV) with R₁ = H, R², R³, R⁴ and R⁵, each independently of one another = CH₃, R⁶ = linear C₁₂ alkyl group, X⁻ = chloride, A = NH, m, n and o each independently of one another = 3,
⁷⁾copolymer of N-vinylpyrrolidone and vinyl acetate.

In embodiments AF1 to AF48, the at least one anionic cross-linked polymer (AP) preferably has a degree of neutralization of from about 80 to about 100%, that is, from about 80 to about 100% of the anionic groups of the polymer are neutralized with at least one neutralizing agent, in particular an alkanolamine. Furthermore, these embodiments preferably have a viscosity of from about 3,500 to about 5,000 mPa*s, measured with Brookfield RVDV II+ with Heilpath, spindle 4, about 20 rpm, about 20° C.

Despite their gel-like consistency or high viscosity, these embodiments are distinguished by an excellent ability to be sprayed and, after application to keratin fibers, in particular human hair, result in excellent long-term hold and in a high volume effect of the keratin fibers treated with these cosmetic agents. In addition, the cosmetic agents used as contemplated herein lead to an improved hair feel on dry hair and to improved styling properties.

In addition, a further subject of the present disclosure is a method for the temporary deformation of keratin fibers, wherein the method comprises the following method steps:
A. spraying the cosmetic product, as described above, on the keratin fibers,
B. distributing the cosmetic product applied in step A. to the keratin fibers and shaping the keratin fibers into the desired shape.

In the context of the method as contemplated herein, it can be provided that the shaping of the keratin fibers in method step B) is performed at ambient temperature and/or at a temperature of at least about 40° C., in particular of at least about 50° C. Ambient temperature here is understood to mean the temperature which exists without the use of heat sources, such as hot air blow dryers, flat irons or heat hoods, or cold sources. If the deformation of the keratin fibers, in particular of the human hair, is to be performed at higher temperatures of at least about 40° C., it is preferred as contemplated herein when the deformation is performed using a hot air dryer, a smoothing iron, a heat hood, etc. The heating of the keratin fibers may take place during the deformation. However, it can also be provided to heat the keratin fibers after the application and/or after the distribution of the cosmetic agent as contemplated herein.

With regard to further preferred embodiments of the method as contemplated herein, mutatis mutandis applies to the cosmetic agents used as contemplated herein and to the cosmetic product as contemplated herein.

Finally, a further subject of the present disclosure is the use of a cosmetic product as contemplated herein for the temporary deformation of keratin fibers.

With regard to further preferred embodiments of the use as contemplated herein, the statements made for the cosmetic product as contemplated herein and for the method as contemplated herein apply mutatis mutandis.

The following examples illustrate the present disclosure without, however, limiting it thereto:

### EXAMPLES

### 1. Formulations (All Specifications in Percent by Weight, Based on the Total Weight of the Respective Cosmetic Agent)

| Raw material | V1 | E1 |
|---|---|---|
| demineralized water | Ad 100 | Ad 100 |
| Dye | 0.0002 | 0.0002 |
| Phenoxyethanol, pure | 0.5 | 0.5 |
| DMDM Hydantoin 55% | 0.1 | 0.1 |
| D-Panthenol 75% | 0.2 | 0.2 |
| AMP-ULTRA PC 2000¹⁾ | 0.2 | 0.15 |
| Xiameter OFX-0193 Fluid²⁾ | 0.4 | — |
| Abil B 8843³⁾ | 1.0 | — |
| Nonionic polymer (NP)⁴⁾ | 2.0 | 3.5 |
| Luviquat Supreme AT 1 (PQ-68) | 5.0 | — |
| 20%⁵⁾ | | |
| Castor oil hydrog., 40 EO | 0.3 | 0.3 |
| Perfume New Look | 0.15 | 0.15 |
| demineralized water | 0.1 | — |
| Aculyn 22 (30%)⁶⁾ | 2.0 | — |
| Anionic cross-linked polymer (AP)⁷⁾ | — | 0.35 |
| Cationic polymer (KP)⁸⁾ | — | 0.6 |

¹⁾INCI name: Aminomethyl propanol (Dow Corning)
²⁾Silicone polyether copolymer having (CH₂)₃(EO)₁₂OH units, available from Dow Corning ® as 193C fluid, INCI name: PEG-12 Dimethicone
³⁾Silicone surfactant, INCI name: PEG-14 Dimethicone, available as ABIL ® B 8843 from Degussa
⁴⁾Copolymer of N-vinylpyrrolidone and vinyl acetate
⁵⁾INCI name: Polyquaternium-68 (19 to 21% dispersion in water, BASF)
⁶⁾INCI name: Acrylates/Steareth-20 Methacrylate Copolymer (29.5 to 30.5% dispersion in water, Rohm & Haas)
⁷⁾containing 40-55% by weight N-vinylpyrrolidone, 40-55% by weight acrylic acid and 0.8-1.5% by weight pentaerythritol triallyl ether, each based on the total weight of all monomers of the anionic polymer (AP)
⁸⁾containing structural units of formulas (I) to (IV) with R₁ = H, R², R³, R⁴ and R⁵, each independently of one another = CH₃, R⁶ = linear C₁₂ alkyl group, X⁻= chloride, A = NH, m, n and o each independently of one another = 3,

The cosmetic agents V1 and E1 were obtained by mixing the above ingredients and each filled into a non-aerosol container with spray valve. Subsequently, the resulting aerosol sprays and non-aerosol sprays were applied to keratin fibers. Comparative example Cl shows an insufficient spreadability of the gel and results in sticky-feeling hands after use. The product El as contemplated herein, despite a viscosity of 4,000 mPa*s, measured with Brookfield RVDV II+ with Heilpath, spindle 4, 20 rpm, 20° C., can be excellently sprayed, does not lead to a blockage of the valve and has a high hold and a good hair feel.

The application properties were tested by a panel of five hairdressers and scored on points (0 points: very bad, highest point score: 6).

The mean values which result when using the formulation according to El are compared with the results of formulation V1 (in brackets). The hold resulted in a value of 5.00 (4.40). The degree of hold level achieved for the product was 3.20 (2.40). The hair feel of the dry hair was 5.00 (4.20). The volume was 4.80 (4.40). The fullness of the hair was 5.00 (4.20). The styling properties with respect to a desired shape were 4.80 (4.20).

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic product comprising
A. a dispensing device with spray valve,
B. a container connected to the dispensing device, comprising a cosmetic agent comprising
b1) at least one cross-linked anionic polymer (AP) comprising at least the following monomers:
bb1) at least one vinylamide monomer A selected from the group of N-vinylpyrrolidone, N-vinylcaprolactam, N-vinylformamide, N-vinylacetamide and N-vinyl-N-methylacetamide,
bb2) at least one acrylic monomer B selected from the group of acrylic acid, methacrylic acid, C₁-C₆ alkyl acrylates, C₁-C₆ alkyl methacrylates, crotonic acid, itaconic acid and maleic acid
bb3) at least one acrylic monomer C having at least 2 vinyl groups, selected from the group of pentaerythritol triallyl ether, pentaerythritol triacrylate, pentaerythritol tetraacrylate and methylenebisacrylamide,
b2) at least one cationic polymer (KP) comprising at least one structural unit of the formula (I), at least one structural unit of the formula (II) and at least one structural unit of the formula (III) 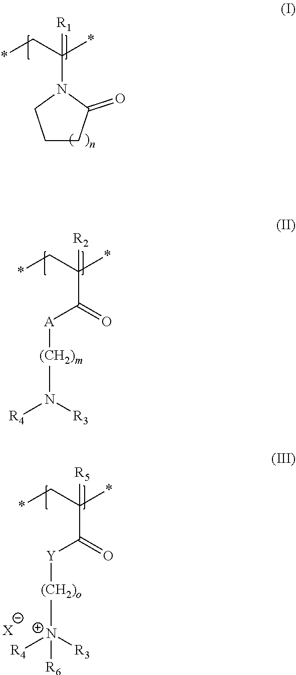
wherein
R¹, R² and R⁵, each independently of one another, stand for hydrogen or a C₁-C₄ alkyl group.
n, m and o, each independently of one another, stand for integers from about 1 to about 4,
A and Y, each independently of one another, represent an oxygen atom, a sulfur atom or an NH group,
R³ and R⁴, each independently of one another, stand for hydrogen or a C₁-C₄ alkyl group,
X⁻ stands for a physiologically compatible anion, and
b3) at least one nonionic polymer (NP),
b4) propellant in a total amount of from about 0 to about 95% by weight, based on the total weight of the cosmetic agent found in the container.

2. The cosmetic product according to claim 1, wherein the at least one cross-linked anionic polymer (AP) has a degree of neutralization of from about 40 to about 100%.

3. The cosmetic product according to claim 1, wherein the cosmetic agent comprises the at least one cross-linked anionic polymer (AP) in a total amount of from about 0.1 to about 1.0% by weight based on the total weight of the cosmetic agent.

4. The cosmetic product according to claim 1, wherein the cationic polymer (KP) additionally comprises at least one structural unit of the formula (IV)

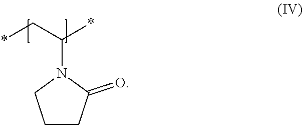

5. The cosmetic product according to claim 1, wherein the cosmetic agent comprises the at least one cross-linked cationic polymer (AP) in a total amount of from about 0.5 to about 5.0% by weight based on the total weight of the cosmetic agent.

6. The cosmetic product according to claim 1, whreein the at least one nonionic polymer (NP) is selected from the group of homopolymers of polyvinylpyrrolidone, homopolymers of polyvinyl acetate, homopolymers and copolymers of polyvinyl alcohol, copolymers of N-vinylpyrrolidone and vinyl acetate mixtures thereof.

7. The cosmetic product according to claim 1, wherein the cosmetic agent comprises the at least one nonionic polymer (NP) in a total amount of from about 0.5 to about 5.0% by weight, preferably from 06 to 4 0% by based on the total weight of the cosmetic agent

8. The cosmetic product according to claim 1, wherein the cosmetic agent additionally comprises at least one alkalizing agent selected from the group of organic and inorganic bases.

9. (canceled)

10. A method for the temporary deformation of keratin fibers, wherein the method comprises the following method steps:
A. spraying the cosmetic product according to claim 1 on the keratin fibers,
B. distributing the cosmetic product applied in step A. to the keratin fibers and shaping the keratin fibers into the desired shape.

11. The cosmetic product according to claim 1 wherein the at least one vinylamide monomer A is N-vinylpyrrolidone.

12. The cosmetic product according to claim 1 wherein the at least one acrylic monomer B is acrylic acid.

13. The cosmetic product according to claim 1 wherein the at least one acrylic monomer C is pentaerythritol triallyl ether.

14. The cosmetic product according to claim 1 wherein A and Y each represent NH group.

15. The cosmetic product according to claim 1 wherein R³ and R⁴ each stand for a methyl group.

16. The cosmetic product according to claim 1 wherein R⁶ stands for a C₁₂ alkyl group.

17. The cosmetic product according to claim 1 wherein X⁻ stands for chloride.

18. The cosmetic product according to claim 1 wherein the at least one vinylamide monomer A is N-vinylpyrrolidone, wherein the at least one acrylic monomer B is acrylic acid, wherein the at least one acrylic monomer C is pentaerythritol triallyl ether, wherein A and Y each represent an NH group, wherein R³ and R⁴ each stand for a methyl group, wherein R⁶ stands for a C₁₂ alkyl group, and wherein X⁻ stands for chloride.

19. The cosmetic product according to claim 1, wherein the at least one cross-linked anionic polymer (AP) has a degree of neutralization of from about 80 to about 100%.

20. The cosmetic product according to claim 1, wherein the cosmetic agent comprises the at least one cross-linked anionic polymer (AP) in a total amount of from about 0.2 to about 0.4% by weight, based on the total weight of the cosmetic agent.

21. The cosmetic product according to claim 1 wherein the at least one vinylamide monomer A is N-vinylpyrrolidone, wherein the at least one acrylic monomer B is acrylic acid, wherein the at least one acrylic monomer C is pentaerythritol triallyl ether, wherein A and Y each represent an NH group, wherein R³ and R⁴ each stand for a methyl group, wherein R⁶ stands for a C₁₂ alkyl group, wherein X⁻ stands for chloride, wherein the at least one cross-linked anionic polymer (AP) has a degree of neutralization of from about 80 to about 100%, wherein the cosmetic agent comprises the at least one cross-linked anionic polymer (AP) in a total amount of from about 0.2 to about 0.4% by weight, based on the total weight of the cosmetic agent, wherein the cationic polymer (KP) additionally comprises at least one structural unit of the formula (IV) 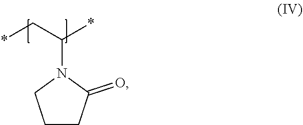
wherein the at least one cross-linked cationic polymer (AP) is present in a total amount of from about 1.5 to about 2.5% by weight, based on the total weight of the cosmetic agent, wherein the at least one nonionic polymer (NP) is selected from N-vinylpyrrolidone and vinyl acetate, and mixtures thereof, wherein the copolymers of N-vinylpyrrolidone and vinyl acetate, are present in a total amount of from about 1.0 to about 2.0% by weight, based on the total weight of the cosmetic agent, and wherein the cosmetic agent additionally comprises at least one alkalizing agent selected from the group of alkanolamines.
